(19) United States
(12) Patent Application Publication
(10) Pub. No.: US 2023/0113110 A1
(43) Pub. Date: Apr. 13, 2023

(54) COMPOSITION FOR THE TREATMENT OF LESIONS OF THE RESPIRATORY SYSTEM

(71) Applicants: Denis BARRITAULT, PARIS (FR); ORGANES,TISSUS: REGENERATION, REPARATION, REMPLACEMENT, PARIS (FR)

(72) Inventor: Denis BARRITAULT, PARIS (FR)

(21) Appl. No.: 17/910,757

(22) PCT Filed: Mar. 8, 2021
(86) PCT No.: PCT/FR2021/050385

§ 371 Date: Sep. 9, 2022

(30) Foreign Application Priority Data
Mar. 9, 2020 (WO) PCT/FR2020/000052
Aug. 24, 2020 (WO) PCT/EP2020/073649

(51) Int. Cl. A61K 31/795; A61K 31/728; A61P 31/16

(52) U.S. Cl. CPC A61K 31/795; A61K 31/728; A61P 31/16

(57) ABSTRACT

The present invention relates to a pharmaceutical composition for use in the prophylaxis and/or treatment of lesions of the respiratory system, in particular lung lesions, caused by a microorganism.

The present invention also relates to a pharmaceutical composition for use in the treatment of lesions of the respiratory system, in particular lung lesions, caused by a microorganism.

The present invention has an application in particular in the therapeutic, pharmaceutical and veterinary fields.

### TECHNICAL FIELD

The present invention relates to a pharmaceutical composition for use in the prophylaxis and/or treatment of lesions of the respiratory system, in particular lung lesions, caused by a microorganism.

The present invention also relates to a pharmaceutical composition for use in the treatment of lesions of the respiratory system, in particular lung lesions, caused by a microorganism.

The present invention has an application in particular in the therapeutic, pharmaceutical and veterinary fields.

In the following description, the references between parentheses () refer to the list of references presented at the end of the text.

### STATE OF THE ART

Tissue lesions, particularly of the respiratory system can occur and/or be caused by many factors, for example air pollution, microorganisms, for example viruses, bacteria, fungi. Pathologies can also be the cause of lesions of the respiratory system, for example bronchitis, pneumonia, tuberculosis, chronic obstructive pulmonary disease (COPD), pulmonary fibrosis, bronchopulmonary cancer.

Different treatments are known according to the pathologies of the respiratory system. For example, the administration of inhaled corticosteroids can be used to reduce, for example, the inflammation of the bronchi, the administration of antibiotics is also used for pathologies related to bacteria, for example in the case of pneumonia.

However, these treatments may have relative degrees of efficacy. Furthermore, in the case of pathologies involving lesions of the pulmonary system, there is no treatment and/or means to treat these lesions.

Lung infections also referred to as pneumopathies can be caused by a microorganism, in particular a virus. The symptoms of a pneumopathy are variable according to the microorganism. For example, they can cause high fever, for example close to 40 degrees, chest pain, cough, fatigue, shortness of breath etc. Lung and/or respiratory system infections are known to be contagious, in particular if they are caused by a microorganism, for example a bacterium or a virus.

The treatment of lung infections depends on the infectious microorganism. In the case of bacterial lung infections, the known treatments involve the use of antibiotics. In the case of viral lung infections, such as influenza, there are no specific treatments for these infections, except for antiviral treatments, which have relative degrees of efficacy or are inefficacious.

In the state of the art there are compounds used in the therapeutic field that can improve the tissue environment, for example biocompatible polymers that are also known to have soothing and pain-relieving properties, optionally with anti-fibrosis activities. Compounds, particularly sulfated polyanions like oligosaccharides, carrageenan, cellulose sulfate, naphthalene sulfonates. However, these compounds have failed to show efficacy, particularly in the treatment of respiratory system infections.

There is therefore a real need in the state of the art for a compound and/or a composition that makes it possible to improve the treatment of infections of the respiratory system caused by a microorganism, in particular lung infections caused by a microorganism.

There is therefore a real need in the state of the art for a compound and/or a composition that makes it possible to improve the treatment of infections of the respiratory system caused by a virus, in particular lung infections caused by a virus.

There is additionally a real need in the state of the art for a compound and/or a composition that makes it possible to improve the treatment of lesions of the respiratory system caused by a microorganism, in particular lung infections caused by a microorganism.

There is also a real need in the state of the art for a compound and/or a composition that makes it possible to improve the treatment of lesions of the respiratory system caused by a virus, in particular lung infections caused by a virus.

### DESCRIPTION OF THE INVENTION

The present invention aims specifically to address these needs by providing a pharmaceutical composition for use in the treatment of lesions of the respiratory system caused by a microorganism, preferably lung lesions caused by a microorganism, said composition comprising
a biocompatible polymer of the following general formula (I)

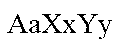

wherein:
   A is a monomer,
   X is an R₁COOR₂ or -R₉(C=O)R₁₀ group,
   Y is an O or N-sulfonate group and has one of the following formulas -R₃OSO₃R₄, -R₅NSO₃R₆, R₇SO₃R₈ wherein:
   R₁, R₃, Rs and R₉ are independently an aliphatic hydrocarbon chain, optionally branched and/or unsaturated and optionally containing one or more aromatic rings with the exception of benzylamine and benzylamine sulfonate, R₂, R₄, R₆ and R₈ are independently a hydrogen atom or an M⁺ cation,
   R₇ and R₁₀ are independently a bond, an aliphatic hydrocarbon chain, optionally branched and/or unsaturated,
   a is the number of monomers,
   x is the rate of substitution of the A monomers by X groups,
   y is the rate of substitution of the A monomers by Y groups.

The present invention also aims to meet these needs by providing a pharmaceutical composition for use in the prophylaxis of lesions of the respiratory system caused by a microorganism, preferably lung lesions caused by a microorganism, said composition comprising
a biocompatible polymer of the following general formula (I)

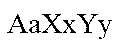

wherein:
   A is a monomer,
   X is an R₁COOR₂ or -R₉(C=O)R₁₀ group,
   Y is an O or N-sulfonate group and has one of the following formulas -R₃OSO₃R₄, -R₅NSO₃R₆, R₇SO₃R₈ wherein:
   R₁, R₃, Rs and R₉ are independently an aliphatic hydrocarbon chain, optionally branched and/or unsaturated and optionally containing one or more aromatic rings with the exception of benzylamine and benzylamine sulfonate, R₂, R₄, R₆ and R₈ are independently a hydrogen atom or an M⁺ cation,
   R₇ and R₁₀ are independently a bond, an aliphatic hydrocarbon chain, optionally branched and/or unsaturated,
   a is the number of monomers,
   x is the rate of substitution of the A monomers by X groups,
   y is the rate of substitution of the A monomers by Y groups.

Advantageously, the inventor has demonstrated surprisingly that the use of biocompatible polymer according to the invention, in particular the composition comprising a biocompatible polymer according to the invention makes it possible advantageously to treat lesions of the respiratory system caused by a microorganism. In particular, the inventor has demonstrated surprisingly and unexpectedly that the composition according to the invention makes it possible advantageously in a synergistic manner to repair the lesions of the pulmonary tissues caused by microorganisms, in particular and advantageously by viral infections.

Additionally, the inventor has demonstrated surprisingly and unexpectedly that the composition according to the invention makes it possible advantageously to repair lesions of the pulmonary tissues in a very short time and also advantageously and unexpectedly enables a functional recovery of the tissues and/or organs of the damaged respiratory system. The inventor has also demonstrated that the composition according to the invention advantageously enables a recovery of the function of the alveolar-capillary barrier or air-blood barrier. In particular, the inventors have demonstrated that the composition according to the invention makes it possible advantageously to neutralize the effects of infections of the respiratory system, in particular of infections of the pulmonary system, in particular of viral infections, through functional respiratory recovery, namely a return to a normal functioning of the pulmonary system, particularly obtained in a short time, for example a few days after the treatment. Additionally, the inventor has demonstrated that the composition according to the invention advantageously enables a repair of pulmonary tissue lesions without relapse.

The inventor has also demonstrated surprisingly and unexpectedly that the composition according to the invention advantageously and unexpectedly enables protection of the alveolar-capillary barrier or air-blood barrier. In particular, the inventors have demonstrated that the composition according to the invention can advantageously be used prophylactically in patients exposed to at least one microorganism causing lesions of the respiratory system and/or in patients with lesions of the respiratory system caused by a microorganism. In particular, the inventor has demonstrated that the composition according to the invention makes it possible advantageously to neutralize the effects of infections of the respiratory system caused by a microorganism, in particular of infections of the pulmonary system, in particular of viral infections, and thus makes it possible to use the composition according to the invention prophylactically, for example before any symptom, for example respiratory. This effect could be observed in an environment

The inventor has also demonstrated that the composition enables a rapid recovery of the respiratory functions impaired by lung lesions caused by a microorganism and advantageously a recovery of the pulmonary function, in particular a restoration of the function of the alveolar-capillary barrier or air-blood barrier.

The present invention thus also relates to a pharmaceutical composition for use in the treatment of deficiencies of respiratory functions due to lesions of the respiratory system caused by a microorganism, preferably lung lesions caused by a microorganism, said composition comprising
a biocompatible polymer of the following general formula (I)

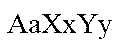

wherein:
   A is a monomer,
   X is an R₁COOR₂ or -R₉(C=O)R₁₀ group,
   Y is an O or N-sulfonate group and has one of the following formulas -R₃OSO₃R₄, -R₅NSO₃R₆, R₇SO₃R₈ wherein:
   R₁, R₃, Rs and R₉ are independently an aliphatic hydrocarbon chain, optionally branched and/or unsaturated and optionally containing one or more aromatic rings with the exception of benzylamine and benzylamine sulfonate, R₂, R₄, R₆ and R₈ are independently a hydrogen atom or an M⁺ cation,
   R₇ and R₁₀ are independently a bond, an aliphatic hydrocarbon chain, optionally branched and/or unsaturated,
   a is the number of monomers,
   x is the rate of substitution of the A monomers by X groups,
   y is the rate of substitution of the A monomers by Y groups.

In the present, lesions of the respiratory system caused by a microorganism means any lesion of the respiratory system caused by a microorganism known to a skilled person. These can be for example lesions of the pharynx, lesions of the larynx, lesions of the trachea, lesions of the lungs, lesions of the bronchi and/or lesions of bronchioles caused by a microorganism.

In the present, lung lesions caused by a microorganism means any lung injury caused by a microorganism known to a skilled person. These can be for example lesions of lesions of the lungs, lesions of the bronchi and/or lesions of bronchioles caused by a microorganism. These can be for example respiratory complications following an infection by a microorganism, for example a virus, a bacterium, a fungus, a parasite. These can be for example respiratory complications and/or respiratory effects caused by a microorganisms, for example as described in Aleksandra Milewska et al., “Human Coronavirus NL63 Utilizes Heparan Sulfate Proteoglycans for Attachment to Target Cells” November 2014 Volume 88 Number 22 Journal of Virology pages 13221-13230, De Haan et al, “Cleavage of Group 1 Coronavirus Spike Proteins: How Furin Cleavage Is Traded Off against Heparan Sulfate Binding upon Cell Culture Adaptation”, JOURNAL OF VIROLOGY, pages 6078-6083 Vol. 82, June 2008.

In the present, the terms “treatment”, “cure”, “treated” or “treat” refer to prophylaxis and/or therapy, in particular when the aim is to prevent and/or slow down (reduce) a lesion of the respiratory system caused by a microorganism, preferably a virus, and/or a lung lesions caused by a microorganism, for example a microorganism selected from the group consisting of a virus, a bacterium, a parasite and a fungus, preferably a virus. Beneficial or desired clinical outcomes comprise, but are not limited to, alleviation of symptoms, reduction of the scope of the disease, stabilization (i.e., non-aggravation) of the disease state, delay or slowing of disease progression, improvement or palliation of the disease state, and remission (partial or complete), whether detectable or not. “Treatment” can also mean prolongation of survival and/or improvement in the quality of life compared to the survival and/or the quality of life expected if the patient does not receive treatment. Advantageously, the treatment may comprise one of the following: reduction in the respiratory syndrome, reduction in respiratory distress, reduction in lung pain, reduction in breathing difficulties and/or pain, reduction in cough frequency.

In the present, prophylaxis means particularly the prevention of impairments and/or lesions of the respiratory system caused by a microorganism, for example a microorganism selected from the group comprising a virus, a bacterium, a parasite and a fungus, preferably a virus.

In the present, prophylaxis also means any degree of delay in the onset of clinical signs or symptoms of lesions of the respiratory system as well as any degree of inhibition of the severity of clinical signs or symptoms of lesions of the respiratory system, including, but not limited to, the total prevention of lesions of the respiratory system. For example, prophylaxis can comprise administration of a composition according to the invention to a mammal, preferably a human being, likely to be colonized and/or infected by a microorganism likely to cause lung lesions, for example, as a preventive measure, that is to say in order to prevent colonization by said microorganism or to avoid the appearance of any clinical sign or symptom of lung lesions. Prophylactic administration can be carried out before said mammal, preferably human being, is exposed to an organism likely to cause lung lesions in said mammal (in particular in said human) or at the time of exposure. Such prophylactic administration can make it possible advantageously to prevent, ameliorate, and/or reduce the severity of any subsequent lung injury. Thus advantageously from the first signs such as throat irritation, coughing, repeated sneezing are advantageously controlled by an administration, for example by inhalation, of the composition.

In the present, respiratory functions mean ventilation and the exchange of oxygen (O₂) and carbon dioxide (CO₂) between the air and the blood, in the pulmonary alveoli. This can be for example the pulmonary function associated with the function of the alveolar-capillary barrier or air-blood barrier involved in the gas exchange, in particular of oxygen (O₂) and carbon dioxide (CO₂) between the air and the blood.

In the present, deficiency of the respiratory functions means a reduction and/or impairment of the exchange of oxygen (O₂) and carbon dioxide (CO₂) between the air and the blood, in the pulmonary alveoli. It can be for example a deficiency of respiratory functions likely to induce respiratory acidosis. This can be manifested clinically by difficult breathing, gasping, shortness of breath and a feeling of suffocation, sometimes painful in the chest, and great fatigue. The skin on the fingers and lips may turn blue.

In the present, microorganism means any microorganism known to the skilled person that is likely to cause lesions of the respiratory system and/or lung lesions. It can be for example a microorganism selected from the group comprising viruses, bacteria, fungi and parasites

In the present, virus means any virus known to the skilled person likely to cause lesions of the respiratory system and/or lung lesions. It can be for example a virus selected from the group comprising the Picornavirus family, for example rhinoviruses, the Coronaviridae family, for example coronaviruses, the Orthomyxoviridae family, for example influenza viruses. It can be for example a virus selected from the group comprising coronaviruses, rhinoviruses, influenza viruses. It can be for example viruses, in particular viruses of the Coronaviridae family, selected from the group comprising coronavirus 229E, coronavirus NL63 (HCoV-NL63) (human coronavirus NL63), human coronavirus OC43 (HCoV-OC43), human coronavirus HKU1 ((HCoV-HKU1), acronym, SARS coronavirus (severe acute respiratory syndrome), MERS-CoV coronavirus (Middle East Respiratory Syndrome Coronavirus), SARS-CoV-1 coronavirus (severe acute respiratory syndrome coronavirus), SARS-CoV-2 coronavirus (severe acute respiratory syndrome coronavirus 2). It can be for example viruses, in particular viruses of the Picornavirus family, selected in the group comprising human rhinovirus C, human rhinovirus B, human rhinovirus A. It can be for example viruses, in particular viruses of the Orthomyxoviridae family, selected from the group comprising influenza A viruses, influenza B viruses, influenza C viruses.

In the present, the inventors have demonstrated that the composition according to the invention can make it possible advantageously to treat lesions of the respiratory system and/or lung lesions of viruses using heparan sulfates as a gateway to infest the target cell, for example as described in the document Cagno et al. “Heparan Sulfate Proteoglycans and Viral Attachment: True Receptors or Adaptation Bias?” Viruses. 2019 Jul; 11(7): 596.

In the present, it can be for example a virus selected from the group comprising Dengue viruses (DENV), Echoviruses 5, for example metapneumovirus, rhinoviruses, enteroviruses, human immunodeficiency virus (HIV), the Zika virus, the Chikungunya virus. It can be any virus known to the skilled person that is likely to use heparan sulfates as co-receptors for entry into cells

In the present, bacteria means any bacterium known to the skilled person likely to cause lesions of the respiratory system and/or lung lesions. It can be for example a bacterium selected from the group comprising Streptococcus pneumoniae, Hemophilus influenza type B or a mycoplasma for example Mycoplasma pneumoniae

In the present, fungus means any fungus known to the skilled person likely to cause lesions of the respiratory system and/or lung lesions. It can be for example a fungus selected from the group comprising Pneumocystis jirovici, Cryptococcus neoformens, Aspergillus sp and nemathelminthes.

In the present, parasite means any parasite known to the skilled person likely to cause lesions of the respiratory system and/or lung lesions. It can be for example a parasite involved in respiratory pathologies, for example Chagas disease, pulmonary amoebiasis. It can be for example a parasite selected from the group comprising Trypanosoma cruzi or amoebae, for example Entamoeba histolytica.

In the present, monomer means for example a monomer selected from the group consisting of sugars, esters, alcohols, amino acids or nucleotides or derivatives thereof.

In the present invention, the A monomers constitute the basic elements of the polymers of formula I and can be identical or different.

In the present invention, the identical or different A monomers can be independently selected from sugars or derivatives thereof.

In the present invention, the A monomers can be independently monomers of the following formula:

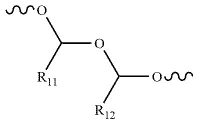

wherein R₁₁ and R₁₂ are independently an oxygen atom, an aliphatic hydrocarbon chain, optionally branched and/or unsaturated, a heteroaryl group comprising independently one or more oxygen and/or nitrogen atoms, an aldehyde function, a carboxylic acid group, a diol, a substituted diol, a group of formula -R₁₃-(X)n-R₁₄ wherein R₁₃ is a C₁ to C₄ aliphatic carbon chain, optionally branched and/or unsaturated, X is a heteroatom selected from oxygen and nitrogen, is an integer ranging from 1 to 4 and R₁₄ is a hydrogen atom, an aliphatic hydrocarbon chain, optionally branched and/or unsaturated, a heteroaryl group comprising independently one or more oxygen and/or nitrogen atoms, an aldehyde function, a carboxylic acid group, a diol, a substituted diol.

In the present invention, the combination of monomers can make it possible to form a polymeric backbone, for example a polymeric backbone of polyester, polyalcohol, polysaccharide, of the nucleic acid or protein type.

In the present invention, among the polyesters, these can be for example copolymers from biosynthesis or chemical synthesis, for example aliphatic polyesters or polyesters of natural origin for example polyhydroxyalkanoates.

In the present invention, the polysaccharides and their derivatives can be of bacterial, animal, fungal and/or plant origin. They can be for example single-chain polysaccharides, for example polyglucoses, for example dextran, cellulose, beta glucan, or other monomers comprising more complex units, for example xanthans, for example glucose, mannose and glucuronic acid, or glucuronans and glucoglucuronan.

In the present invention, the polysaccharides of plant origin can be single-chain, for example cellulose (glucose), pectins (galacturonic acid), fucans, starch or more complex like alginates (galuronic and mannuronic acid).

In the present invention, the polysaccharides of fungal origin can be for example steroglucan.

In the present invention, the polysaccharides of animal origin can be for example chitins or chitosan (glucosamine).

In the present invention, the A monomers that constitute the basic elements of the polymers of formula I can be advantageously identical.

In the present invention, the A monomers that constitute the basic elements of the polymers of formula I can be advantageously glucose.

The number of A monomers defined in formula (I) by “a” can be such that the weight of said polymers of formula (I) is greater than or equal to 2000 Daltons. The number of A monomers defined in formula (I) by “a” can be such that the weight of said polymers of formula (I) is between around 2000 and 6000 Daltons, for example which corresponds to at least 10 glucose monomers. For example, the weight of said polymers of formula (I) can be between around 3000 Daltons and 6000 Daltons, for example which corresponds to 12 to 20 glucose monomers.

The number of A monomers defined in formula (I) by “a” can also be such that the weight of said polymers of formula (I) is less than about 2500000 Daltons (which corresponds to 7000 glucose monomers).

The number of A monomers defined in formula (I) by “a” can also be such that the weight of said polymers of formula (II) can be between around 2000 and 500000 Daltons, for example between 3000 and 500000 Daltons, for example equal to 3000 Daltons, 5000 Daltons, 6000 Daltons, 10000 Daltons, 20000 Daltons, 40000 Daltons, 80000 Daltons, 220000 Daltons, 500000 Daltons.

Advantageously, the weight of said polymers of formula (I) can be comprised from 3000 to 250000 Daltons, for example from 3000 to 6000 Daltons, or for example from 20000 to 250000 Daltons, or for example from 75000 to 150000 Daltons.

Advantageously, the weight of said polymers of formula (I) can be comprised from 3000 to 500000 Daltons, for example from 3000 to 250000 Daltons, for example from 3000 to 6000 Daltons, or for example from 20000 to 250000 Daltons, or for example from 75000 to 150000 Daltons.

In the present invention, in the -R₁COOR₂ group representing X, R₁ can be a C₁ to C₆ alkyl, for example a methyl, ethyl, butyl, propyl, pentyl, preferably a methyl group, and R₂ can be a bond, a C₁ to C₆ alkyl, for example a methyl, ethyl, butyl, propyl, pentyl, an R₂₁R₂₂ group wherein R₂₁ is an anion and R₂₂ a cation selected from the group of the alkali metals.

Preferably, the X group is the group of formula -R₁COOR₂ wherein R₁ is a methyl group -CH₂- and R₂ an R₂₁R₂₂ group wherein R₂₁ is an anion and R₂₂ a cation selected from the group of the alkali metals, preferably the X group is a group of formula -CH₂-COO⁻ or carboxymethyl.

In the present invention, in the -R₉(C=O)R₁₀ group representing X, R₉ can be a C₁ to C₆ alkyl, for example a methyl, ethyl, butyl, propyl, pentyl, preferably a methyl group, and R₁₀ can be a bond, a C₁ to C₆ alkyl, for example a methyl, ethyl, butyl, propyl, pentyl, hexyl.

The rate of substitution of all the A monomers by the X groups defined in the general formula (I) by “x” can be comprised from 10 to 150%, from 40 to 80%, and preferably of the order of 50% or 60%.

In the present invention, in the group having one of the following formulas -R₃OSO₃R₄, -R₅NSO₃R₆, -R₇SO₃R₈ and representing the Y group, R₃ can be a bond, a C₁ to C₆ alkyl, for example a methyl, ethyl, butyl, propyl, pentyl, preferably a methyl group, Rs can be a bond, a C₁ to C₆ alkyl, for example a methyl, ethyl, butyl, propyl, pentyl, preferably a methyl group, R₇ can be a bond, a C₁ to C₆ alkyl, for example a methyl, ethyl, butyl, propyl, pentyl, preferably a methyl group, R₄, R₆ and R₈ can be independently a hydrogen atom or a cation M⁺, for example M⁺ can be an alkali metal.

Preferably, the Y group is the group of formula -R₇SO₃R₈ wherein R₇ is a bond and R₈ is an alkali metal selected from the group comprising lithium, sodium, potassium, rubidium and cesium. Preferably, the Y group is a -SO₃⁻, -SO₃⁻ Na⁺ group

The rate of substitution of all the A monomers by the Y groups defined in the general formula (I) by “y” can be comprised from 10 to 170%, from 30 to 150%, from 55 to 160%, from 55 to 85%, from 120 to 160%, and preferably of the order of 70, 140 or 150%.

In the present invention, the definition of the rates of substitution above, a rate of substitution “x” of 100% means that each A monomer of the polymer of the invention statistically contains an X group. Likewise, a rate of substitution “y” of 100% means that each monomer of the polymer of the invention statistically contains a Y group. Rates of substitution greater than 100% reflect the fact that each monomer statistically has more than one group of the considered type; conversely, rates of substitution of less than 100% reflect the fact that each monomer statistically has less than one group of the considered type.

The polymers can also comprise functional chemical groups, denoted Z, other than X and Y

In the present invention, the Z groups can be identical or different, and can be independently selected from the group comprising amino acids, fatty acids fatty alcohols, ceramides or derivatives thereof, nucleotide addressing sequences, antibodies, antibody fragments.

The Z groups can also be identical or different active agents. They can be, for example, therapeutic agents, diagnostic agents, an anti-inflammatory, an antimicrobial, an antibiotic, an antiviral agent, a growth factor, a cellular communication cytokine, for example an interferon, an enzyme, an antioxidant compound, polyphenols, tannins, anthocyanins, lycopenes, terpenoids and resveratrol. In the present invention, the Z group can advantageously be a saturated or unsaturated fatty acid. It can be for example a fatty acid selected from the group comprising acetic acid, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, trans-vaccenic acid, linoleic acid, linolelaidic acid, α-linolenic acid, γ-linolenic acid, dihomo-γ-linolenic acid, arachidonic acid, eicosapentaenoic acid, clupanodonic acid or docosahexaenoic acid. Preferably, the fatty acid is acetic acid.

In the present invention, the Z group can advantageously be an amino acid of the L or D series selected from the group comprising alanine, asparagine, an aromatic chain for example tyrosine, phenylalanine, tryptophan, thyroxine or histidine. Preferably, the amino acid is phenylalanine.

In the present invention, the Z group can be an antioxidant, for example vitamin A, C, E, B9, B6, glutathione, selenium, polyphenols, for example catechins, for example green tea, flavonoids, tannins, anthocyanins, for example fruit red, lycopene, terpenoids and resveratrol.

In the present invention, the Z group can be anti-aging compounds, for example retinoids, allantoins.

In the present invention, the Z group can be antibodies, antibody fragments, for example Fab fragments. It can be for example addressing antibodies and/or antibody fragments, for example antibodies and/or antibody fragments likely to target the blood-brain barrier.

In the present invention, the Z group can be antiviral agents. It can be any suitable antiviral agent, for example an antiviral agent blocking access to virus entry into the cell or acting as receptor decoys and/or receptor mimetics and/or co-receptor or anti-idiotype antibodies mimicking the natural virus as a ligand with respect to its receptor. It can be, for example in the case of coronavirus, angiotensin 2 converting enzyme (ACE) inhibitors or a serine protease inhibitor, for example TMPRSS2 involved as co-receptors for viral entry into cells as described by Aleksandra Milewska et al., “Human Coronavirus NL63 Utilizes Heparan Sulfate Proteoglycans for Attachment to Target Cells” November 2014 Volume 88 Number 22 Journal of Virology pages 13221-13230 and by Hoffmann et al. in “SARS-CoV-2 Cell Entry Depends on ACE2 and TMPRSS2 and Is Blocked by a Clinically Proven Protease Inhibitor 2020”, Cell 181, 1-10 https://doi.org/10.1016/j.ce11.2020.02.052

Advantageously the Z groups can confer to the polymers additional biological or physicochemical properties. For example the Z groups can increase the solubility or the lipophilicity of said polymer enabling for example a better diffusion or tissue penetration.

Advantageously, the Z groups can confer to the polymers additional biological or physicochemical properties Thus, the polymers of the invention, for example when the Z group is selected from an antioxidant compound, an anti-aging compound, the polymers of the invention can advantageously convey these compounds and thus provide an additional and/or complementary biological effect.

Polymers in which Z is present can have the following formula II: Aa Xx Yy Zz (II) wherein, A, X, Y, a, x, y are as defined herein before and z is the rate of substitution by Z groups.

In the present invention, the A monomers that constitute the basic elements of the polymers of formula (II) can advantageously be identical.

In the present invention, the A monomers that constitute the basic elements of the polymers of formula (II) can advantageously be glucose.

The number of A monomers defined in formula (II) by “a” can be such that the weight of said polymers of formula (II) is greater than or equal to 2000 Daltons. The number of A monomers defined in formula (II) by “a” can be such that the weight of said polymers of formula (II) is about 2000 to 6000 Daltons, for example which corresponds to at least 10 glucose monomers. For example the weight of said polymers of formula (II) can be between around 3000 Daltons and 6000 Daltons, for example, which corresponds to 12 to 20 glucose monomers.

The number of A monomers defined in formula (II) by “a” can also be such that the weight of said polymers of formula (II) is less than about 2500000 Daltons (which corresponds to 7000 glucose monomers).

The number of A monomers defined in formula (II) by “a” can also be such that the weight of said polymers of formula (II) can be between around 2000 and 500000 Daltons, for example between 3000 and 500000 Daltons, for example equal to 3000 Daltons, 5000 Daltons, 6000 Daltons, 10000 Daltons, 20000 Daltons, 40000 Daltons, 80000 Daltons, 220000 Daltons, 500000 Daltons.

Advantageously, the weight of said polymers of formula (II) can be comprised from 3000 to 500000 Daltons, for example from 3000 to 250000 Daltons, for example from 3000 to 6000 Daltons, or for example from 20000 to 250000 Daltons, or for example from 75000 to 150000 Daltons.

The rate of substitution of all the A monomers by the X groups defined in the general formula (II) by “x” can be from 10 to 150%, from 40 to 80%, and preferably of the order of 50% or 60%.

The rate of substitution of all the A monomers by the Y groups defined in the general formula (II) by “y” can be from 10 to 170%, from 30 to 150%, from 55 to 160%, from 55 to 85%, from 120 to 160%, and preferably of the order of 70, 140 or 150%.

In the present invention the rates of substitution by Z groups represented by “z” can be from 1 to 50%, from 10 to 25%, preferably equal to 15, 20 or 25%.

The X, Y and Z groups can be independently bonded to the A monomer and/or independently bonded to one another. When at least one of the X, Y and Z groups is independently bonded to an X, Y and Z group other than the first, one of said X, Y or Z groups is bonded to the A monomer.

Thus, the Z groups can be bonded by covalence directly to the A monomers or bonded by covalence to the X and/or Y groups.

In the present invention the Z groups can also be conjugated with the polymers of formula AaXxYy by bonds other than covalent bonds, for example by ionic bonds, for example via ionic interactions, hydrophilic bonds or hydrophobic bonds. The polymers of the invention can then constitute a Z vectorization system.

In the present invention, the polymer can be for example a polymer selected from the group comprising the compounds OTR4120, OTR41201, OTR41202, OTR41203, OTR41205, OTR41210, OTR41301, OTR41302, OTR41303, OTR41305, OTR41310, OTR3131.

In the present document, the polymer can be for example a polymer selected from the group comprising the compounds OTR41201, OTR41202, OTR41203, OTR41205, OTR41210, OTR4120, OTR4122, OTR4125, OTR41301, OTR41302, OTR41303, OTR41305, OTR41310, OTR3131, OTR4132, OTR4135, OTR415 with the characteristics mentioned in table 1 below

TABLE 1

list and characteristics of polymers

| polymer | | A: glucose | X -CH₂COO- | Y -SO₃⁻ | Z -OCCH₃ | Z phenylalanine |
|---|---|---|---|---|---|---|
| Name of the RGTA | Average molecular weight M or mass +/-15% (MW in Dalton) | Dextran starting polymer (MW in Dalton) | % substitution CM (X)/glucose | % substitution -SO₃₋ (Y)/glucose) | % substitution OCCH₃ (Z)/glucose | % substitution phenylalanine (Z)/glucose |
| CMDS OTR41201 | 3 000 | 1 500 | 60+/-20 | 150+/-20 | 0 | |
| CMDS OTR41202 | 6 000 | 3 000 | 60+/-20 | 150+/-20 | 0 | |
| CMDS OTR41203 | 10 000 | 5 000 | 60+/-20 | 150+/-20 | 0 | |
| CMDS OTR41205 | 20 000 | 10 000 | 60+/-20 | 150+/-20 | 0 | |
| CMDS OTR41210 | 40 000 | 20 000 | 60+/-20 | 150+/-20 | 0 | |
| CMDS OTR4120 | 80 000 | 40 000 | 60+/-20 | 150+/-20 | 0 | |
| CMDS OTR4122 | 220 000 | 110 000 | 60+/-20 | 150+/-20 | 0 | |
| CMDS OTR4125 | 500 000 | 250 000 | 60+/-20 | 150+/-20 | 0 | |
| CMDSA OTR41301 | 3 000 | 1 500 | 60+/-20 | 140+/-20 | 20+/-5 | |
| CMDSA OTR41302 | 6 000 | 3 000 | 60+/-20 | 140+/-20 | 20+/-5 | |
| CMDSA OTR41303 | 10 000 | 5 000 | 60+/-20 | 140+/-20 | 20+/-5 | |
| CMDSA OTR41305 | 20 000 | 10 000 | 60+/-20 | 140+/-20 | 20+/-5 | |
| CMDSA OTR41310 | 40 000 | 20 000 | 60+/-20 | 140+/-20 | 20+/-5 | |
| CMDSA OTR4131 | 80 000 | 40 000 | 60+/-20 | 140+/-20 | 20+/-5 | |
| CMDSA OTR4132 | 220 000 | 110 000 | 60+/-20 | 140+/-20 | 20+/-5 | |
| CMDSA OTR4135 | 500 000 | 250 000 | 60+/-20 | 140+/-20 | 20+/-5 | |
| CMDSP OTR415 | 5 000 | | 60+/-20 | 70+/-15 | - | 15+/-5 |

Table 1: Polymers of the Aa Xx Yy (I) and Aa Xx Yy Zz (II) families wherein A is glucose (MW 180 Da), X is CarboxyMethyl (MW 58 Da) Y: SO₃⁻ (MW 80 Da) and Z is Acetate (MW 43 Da) or phenylalanine (MW 165 Da).

In the present invention, the composition can comprise a concentration of 0.1 to 100 µg/mL by weight of biocompatible polymer with respect to the volume of the composition. For example the composition can comprise a concentration of 1 to 100 µg/mL, of 10 to 100 µg/ml by weight of biocompatible polymer with respect to the total volume of the composition.

In the present invention, the composition can be formulated and/or adapted according to its administration. For example, for parenteral administration the composition can be administered so as to deliver a dose of biocompatible polymer comprised from 0.01 to 5 mg per kilogram of body weight, preferably from 0.1 to 1.5 mg per kilogram of body weight with the frequency of one administration, one administration every two or three days, for example 2 to 3 times per week.

For example, for oral administration the composition can be administered so as to deliver a dose of biocompatible polymer comprised from 0.1 to 5 mg per kilogram of body weight, preferably from 0.01 to 1.5 mg/kg with the frequency of one daily or bi-weekly administration.

For sublingual administration, the dose can be daily or bi-weekly and between 0.5 µg/kg and 100 µg/kg.

For example, for intra-arterial administration, the biocompatible polymer can be at a concentration ranging from 0.1 to 100 µg/mL by weight of biocompatible polymer with respect to the total volume of the composition, preferably from 1 to 20 mL.

For example, for oral administration, the biocompatible polymer can be at a concentration ranging from 0.1 to 100 µg/mL by weight of biocompatible polymer with respect to the total volume of the composition, preferably from 1 to 20 mL, preferably equal to 5 mL.

For example, for administration by air, for example as a nasal spray, preferably by inhalation or nebulization, the biocompatible polymer can be at a concentration ranging from 0.1 to 100 µg/mL by weight of biocompatible polymer with respect to the total volume of the composition, preferably 1 to 20 mL, preferably equal to 5 mL. It can be for example a composition, preferably from 1 to 20 mL, of an aqueous solution of OTR4120 at 100 µg/mL or OTR4131 at 10 µg/mL placed in a nebulizer allowing delivery of 1 to 3 mL per minute with an administration time comprised, for example from 5 to 10 minutes. It can be for example a composition, preferably of 1 to 20 mL, of an aqueous solution of OTR4120 from 10 to 100 µg/mL, for example at 10 µg/mL of OTR4120, or of OTR4131 from 10 µg/mL to 100 µg/mL placed in a nebulizer allowing delivery of 0.5 to 3 mL per minute with a duration of administration comprised, for example from 5 to 10 minutes. It can be for example a composition of an aqueous solution of OTR4120 of 10 to 100 µg/mL, for example at 10 µg/mL of OTR4120 placed in a nebulizer allowing delivery of 0.5 mL per minute with a duration of administration of 5 minutes.

According to the invention, the composition may further comprise hyaluronic acid.

In the present, “hyaluronic acid” means any hyaluronic acid known to a skilled person, for example a non-sulfated linear glycosaminoglycan made up of repeating units of D-glucuronic acid and N-acetyl-D-glucosamine. It can be for example hyaluronic acid (HA) in its acid form or salt form (hyaluronate), cross-linked hyaluronic acid HA is a non-sulfated linear glycosaminoglycan made up of repeating units of D-glucuronic acid and N-acetyl-D-glucosamine (Tammi R., Agren UM., Tuhkanen AL., Tammi M. Hyaluronan metabolism in skin. Progress in Histochemistry & Cytochemistry. 29(2):1-81, 1994 [26]). It can be for example hyaluronic acid with average molecular weight fractions of 5000 to 3000000 Daltons, preferably between 50000 and 2000000 Daltons. In the present document, the hyaluronic acid can be obtained by any method known to the skilled person. This can be for example methods described in the journal Hyaluronan fragments: an information-rich system (R. Stern et al., European Journal of Cell Biology 58 (2006) 699-715[27]). It can also be natural or modified hyaluronic acid, commercially available, whatever their designations and/or molecular weight, for example commercial hyaluronic acid selected from Hyactive CPN; Cristalhyal; Nutra HA; Oligo HA; D Factor; Hyaluderm; juvelift; Restylane; Revitacare without this list being exhaustive. It can also be hyaluronic acid marketed by the company Contipro (https://www.contipro.com/portfolio/manufacturer-of-anti-ageing-cosmetic-raw-materials/HyActive″).

In the present, the composition may comprise a concentration of 0.1 to 5% by weight of hyaluronic acid with respect to the total weight of the composition. For example the composition can comprise a concentration of 0.2% to 2.5% by weight of hyaluronic acid with respect to the total weight of the composition.

In the present, the composition can comprise a concentration of 1 to 10 mg/mL by weight of hyaluronic acid with respect to the total volume of the composition.

Advantageously, the inventor has demonstrated that the composition comprising a biopolymer and hyaluronic acid makes it possible to treat the lesions of the respiratory system caused by a microorganism, in particular a virus. Additionally the composition comprising a biopolymer and hyaluronic acid makes it possible advantageously to provide a synergistic effect of the repair of lesions of the respiratory system, advantageously of lung lesions.

In the present, “pharmaceutical composition” means any form of pharmaceutical composition known to a skilled person. In the present document, the pharmaceutical composition can be for example an injectable solution. It can be for example an injectable solution, for example for a local or systemic injection, for example in physiological serum, in injectable glucose solution, in the presence of excipients, for example of dextrans, for example at concentrations known to the skilled person, for example from one microgram to several milligrams per mL. The pharmaceutical composition can be for example a drug intended for oral administration selected from the group comprising a liquid formulation, an oral effervescent dosage form, an oral powder, a multiparticle system, an orodispersible dosage form.

For example, when the pharmaceutical composition is for oral administration, it can be in the form of a liquid formulation selected from the group comprising a solution, a syrup, a suspension, an emulsion. When the pharmaceutical composition is in the form of an oral effervescent dosage form, it can be in a form selected from the group comprising tablets, granules, powders. When the pharmaceutical composition is in the form of an oral powder or a multiparticulate system, it can be in a form selected from the group comprising beads, granules, mini-tablets and microgranules. When the pharmaceutical composition is in the form of an orodispersible dosage form, it can be in a form selected from the group consisting of orodispersible tablets, freeze-dried wafers, thin films, a chewable tablet, a tablet, a capsule or medical chewing gum.

According to the present invention, the pharmaceutical composition can be a pharmaceutical composition for oral, for example buccal and/or sublingual administration, for example selected from the group comprising buccal or sublingual tablets, lozenges, drops, a solution for sprays.

According to the present invention, the pharmaceutical composition can be a pharmaceutical composition for topical, transdermal administration, for example selected from the group comprising ointments, creams, gels, lotions, patches and foams.

According to the present invention, the pharmaceutical composition can be a pharmaceutical composition for respiratory or nasal administration, for example in the form of an aerosol, for example administered with a nebulizer and/or inhaler.

According to the present invention, the composition can be a composition for nasal or nasal respiratory or respiratory administration, for example selected from the group comprising nasal drops, nasal spray, nasal powder, aerosols, for example aerosols and/or compressed gas nasal spray, or nebulizers.

According to the present invention, the pharmaceutical composition can be a pharmaceutical composition suitable for intra-pulmonary administration, for example by intra-pulmonary injection.

Advantageously, when the pharmaceutical composition is adapted for nasal or respiratory administration, it can advantageously be bronchopulmonary.

Preferably the pharmaceutical composition can be a pharmaceutical composition for nasal, nasal respiratory or respiratory administration.

According to the present invention, the pharmaceutical composition can be a pharmaceutical composition for parenteral administration, for example subcutaneous, intramuscular, intravenous, intra-arterial, intracranial, intrathecal administration.

The composition of the present invention may also comprise at least one other active ingredient, particularly another therapeutically active ingredient, for example for simultaneous or separate use or for use spread out over time depending on the galenic formulation used. This other ingredient can be for example an active ingredient used for example in the treatment of opportunistic infections which can develop in a patient having infection of the respiratory system caused by a microorganism, for example by a virus or a bacterium. It can also be pharmaceutical products known to the skilled person, for example antibiotics, anti-inflammatories, antivirals

The composition of the present invention may also comprise at least one other active ingredient, particularly another therapeutically active ingredient, for example for simultaneous or separate use or for use spread out over time depending on the galenic formulation used. This other ingredient can be for example an active ingredient used for example in the treatment of opportunistic infections or a vitamin, for example vitamin C used in high doses, for example 50 to 100 mg/kg/day or an analgesic or an antibiotic or a bronchodilator for example salbutamol, or a corticosteroid for example Methylprednisolone or an antiviral, for example interferon alfa-2b or lopinavir antiviral therapy, etc.

In the present, the administration of the biocompatible polymer and the hyaluronic acid can be simultaneous, successive or concomitant.

According to the invention, at least one of the administrations can be carried out by the topical, oral, respiratory route or by injection, preferably by the respiratory route. The two administrations can be carried out in the same way or differently. For example, the biocompatible polymer and the hyaluronic acid can be administered by the respiratory route. The administration can also be a function of the zone and/or of the biological tissue to be treated.

According to the invention, the composition can be, for example, administered only once.

According to the invention, the composition can further be, for example, administered daily, bi-daily and weekly or less. It can be for example administration once a day, twice a day or less, for example once every other day, or weekly.

According to the invention, and the mode of administration the composition can be, for example, for a saline composition administered daily, bi-daily and weekly or less. It can be for example administered once daily, twice daily or less.

According to the invention, the composition can be, for example, administered over a period of 1 day to 3 months, for example for 2 months, for example for 1 month, for example for one week. For example, the composition can be administered over a period of 1 to 3 weeks, for example with a frequency of administration of every day or every other day.

For example, when the composition is in a form suitable for administration by the respiratory route, the composition can preferably be administered with an administration frequency of every two or three days.

According to the invention, the composition can be, for example, administered daily, bi-daily and weekly. It can be for example administered once daily, twice daily or more. According to the invention, the composition can be, for example, administered over a period of 1 day to 3 months, for example for 2 months. For example, the composition can be administered over a period of 3 months with a frequency of administration of every day.

The inventor has surprisingly demonstrated that the combination of a biocompatible polymer of formula AaXxYy or AaXxYyZz and natural or modified hyaluronic acid advantageously and surprisingly makes it possible to obtain a synergistic effect in the treatment. In particular, the inventor has demonstrated that the effect obtained was both a synergy going beyond the individual effects of each of the compounds and also advantageously an increase in the duration of these effects.

Other advantages may be seen by the person skilled in the art by reading the following examples, illustrated by the appended figures provided by way of illustration.

### EXAMPLES

### Example 1: Use of a Biocompatible Polymer in the Treatment of Patients Infected with Influenza Virus

### A/ Preparation of Biocompatible Polymers

The synthesis of biocompatible polymers, RGTA, is widely described in the prior art, for example in U.S. Pat. No. 7,396,923 entitled “Process for the sulfonation of compounds comprising free hydroxyl groups (OH) or primary or secondary amines” and also in the bibliographic reference Yasunori I. et al., Biomaterials 2011, 32:769e776) and Petit E. et al., Biomacromolecules. 2004 Mar-Apr; 5(2):445-52 [28].

Several types of RGTA are known and described have been used including OTR4120 describes many preclinical and clinical publications (RGTA®-based matrix therapy - A new branch of regenerative medicine in locomotion. Barritault D, Desgranges P, Meddahi-Pellé A, Denoix JM, Saffar JL. Joint Bone Spine. 2017 May;84(3):283-292. doi: 10.1016/j.jbspin.2016.06.012 [29], RGTA® or ReGeneraTing Agents mimic heparan sulfate in regenerative medicine: from concept to curing patients. Barritault D, Gilbert-Sirieix M, Rice KL, Siñeriz F, Papy-Garcia D, Baudouin C, Desgranges P, Zakine G, Saffar JL, van Neck J. Glycoconj J. 2017 Jun;34(3):325-338. doi: 10.1007/s10719-016-9744-5 [2]. The compound OTR4131 is a compound comprising a radical Z which is a fatty acid, namely acetic acid as described in Frescaline G. et al., Tissue Eng PartA. 2013 Jul;19(13-14):1641-53. doi: 10.1089/ten.TEA.2012.0377 [30]), Randomized controlled trial demonstrates the benefit of RGTA® based matrix therapy to treat tendinopathies in racing horses. Jacquet-Guibon S, Dupays AG, Coudry V, Crevier-Denoix N, Leroy S, Siñeriz F, Chiappini F, Barritault D, Denoix JM. PLoS One. 2018 Mar 9;13(3):e0191796. doi: 10.1371/journal.pone.0191796 [31]. Other compounds also described in patent documents US06689741, US2014301972A1 in which Z is an amino acid such as phenylalanine (Heparan sulfate proteoglycans mediate internalization and propagation of specific proteopathic seeds. Holmes BB, DeVos SL, Kfoury N, Li M, Jacks R, Yanamandra K, Ouidja MO, Brodsky FM, Marasa J, Bagchi DP, Kotzbauer PT, Miller TM, Papy-Garcia D, Diamond MI. Proc Natl Acad Sci U S A. 2013 Aug 13;110(33):E3138-47. doi: 10.1073/pnas.1301440110 [32]) or another hydrophobic compound (Structure-activity studies of heparan mimetic polyanions for anti-prion therapies. Ouidja MO, Petit E, Kerros ME, Ikeda Y, Morin C, Carpentier G, Barritault D, Brugere-Picoux J, Deslys JP, Adjou K, Papy-Garcia D. Biochem Biophys Res Commun. 2007 Nov 9;363(1 ):95-100 [33]).

### B/ Effect of a Biopolymer, OTR4120, on the Respiratory Functions of an Asthmatic Patient Having a Viral Infection, in Particular Influenza

In the present example, the effects of the biocompatible polymer according to the invention, RGTA OTR4120 on the improvement of respiration in an asthmatic patient affected by influenza were evaluated. The patient was a 35-year-old woman presenting with influenza infection and having a history of hospitalizations (duration of hospitalization 10 days in intensive care) for complications of the viral infection linked particularly to chronic asthma.

The patient was treated with a biocompatible polymer, namely the compound OTR4120, taking every other day for a week (7 days) 5 mL of a saline solution (CACIPLIQ ®, OTR3 Paris France) at 100 µg/mL of OTR4120 by inhalation. The solution was administered orally by inhalation with an electric nebulizer. The inhalation time was 10 minutes using an Omron-type electric inhaler or the like.

24 hours after the first dose, the clinical signs of distress and/or impairment of the respiratory system were greatly reduced. In particular, the frequency of coughing spells, namely several per minute, rapidly diminished and with it the pain associated with breathing, which was gasping, as well as the shivers that ran through the patient. Body temperature also decreased from 40° C. to a normal temperature of 37° C. within a week, with fatigue also going from exhaustion to gradual recovery.

After one week of treatment, the patient no longer showed any signs of distress and/or impairment of the respiratory system.

All of the improvements, in particular at 24 hours after the first dose, were particularly linked to a treatment of lesions of the respiratory system caused by the influenza virus, advantageously allowing an improvement and recovery of the functions of the respiratory system.

The results were subsequently confirmed during subsequent viral infections by an influenza virus of the aforementioned patient, of a female patient having an identical clinical profile and in infants infected by an influenza virus.

Similarly and surprisingly, 24 hours after the first dose of a composition comprising a biocompatible polymer according to the invention, namely OTR4120, the treated patients displayed a significant reduction in any sign of distress and/or impairment of the respiratory system.

All of the improvements, in particular at 24 hours after the first dose, were particularly linked to a treatment of lesions of the respiratory system caused by the influenza virus, advantageously allowing an improvement and recovery of the functions of the respiratory system.

This example clearly demonstrates that examples of a composition according to the invention comprising a polymer of formula AaXxYy or AaXxYyZz make it possible advantageously to treat and/or to improve treatment of lesions of the respiratory system caused by a microorganism, namely a virus, preferably lung lesions caused by a microorganism, in particular a virus. In particular, this example clearly demonstrates that examples of a composition according to the invention, particularly when administered by the pulmonary route, make it possible to treat lung lesions caused by a virus, particularly the influenza virus.

This example also clearly demonstrates that examples of compositions according to the invention make it possible to treat lung lesions caused by a virus, particularly the influenza virus in a very short time, which can advantageously make it possible to reduce the risk of morbidity linked to the lesions of the respiratory system and/or lungs caused in particular by a virus.

This example also clearly demonstrates, through functional recovery, a recovery of lung function linked particularly to a restoration of the function of the alveolar-capillary barrier or air-blood barrier. In other words, an example of a composition according to the invention makes it possible both to treat lung lesions caused by a microorganism, in particular a virus, and also to restore lung function via restoration of the function of the alveolar-capillary barrier or air-blood barrier. Also, as demonstrated in this example, the present invention goes beyond the simple treatment of lung lesions and advantageously makes it possible, during a deficiency in the pulmonary function, for example respiratory distress, to synergistically restore the pulmonary function.

### Example 2: Use of a Biocompatible Polymer in the Treatment of Patients Infected with the MERS-CoV Virus and Presenting with Respiratory Syndrome

In this example, the composition used was identical to that of the aforementioned example 1, namely the polymer OTR4120 (product CACIPLIQ®, OTR3 Paris France (OTR4120)).

The patients were women presenting with clinical signs of respiratory distress, in particular acute respiratory syndrome with coughing fits, gasping and rapid breathing, body shivers, high fever and exhaustion with pain accompanying breathing and had tested positive for the MERS-CoV virus.

The patients were treated with a biocompatible polymer, namely the compound OTR4120, taking every other day for a week (7 days) 5 mL of a saline solution (CACIPLIQ®, OTR3 Paris France (OTR4120)) at 100 µg/mL of OTR4120 by inhalation. The solution was administered orally by inhalation. The inhalation time was 10 minutes.

Surprisingly, 24 hours after the first dose of a composition comprising a biocompatible polymer according to the invention, namely OTR4120, the treated patients displayed a significant reduction in any sign of distress and/or impairment of the respiratory system.

All of the improvements, in particular at 24 hours after the first dose, were particularly linked to a treatment of lesions of the respiratory system caused by the MERS-CoV virus, advantageously allowing an improvement and recovery of the functions of the respiratory system.

This example clearly demonstrates that examples of a composition according to the invention comprising a polymer of formula AaXxYy or AaXxYyZz make it possible advantageously to treat and/or to improve treatment of lesions of the respiratory system caused by a microorganism, namely a virus, preferably lung lesions caused by a microorganism, in particular a virus. In particular, this example clearly demonstrates that examples of a composition according to the invention, particularly when administered by the pulmonary route, make it possible to treat lung lesions caused by viruses of the Coronaviridae family, particularly the MERS-CoV virus.

This example also clearly demonstrates that examples of a composition according to the invention make it possible to treat lung lesions caused by a virus, particularly a coronavirus, in a very short time which can advantageously make it possible to reduce the risk of morbidity linked to the lesions of the respiratory system and/or lungs caused in particular by viruses of the Coronaviridae family.

This example also clearly demonstrates, through functional recovery, a recovery of lung function linked particularly to a restoration of the function of the alveolar-capillary barrier or air-blood barrier. In other words, an example of a composition according to the invention makes it possible both to treat lung lesions caused by a microorganism, in particular a virus, and also to restore lung function via restoration of the function of the alveolar-capillary barrier or air-blood barrier. Also, as demonstrated in this example, the present invention goes beyond the simple treatment of lung lesions and advantageously makes it possible, during a deficiency in the pulmonary function, for example respiratory distress and/or an impairment of the respiratory system, to synergistically restore the pulmonary function.

### Example 3: Use of a Biocompatible Polymer in the Treatment of Patients Infected with Seasonal Influenza Virus

In this example, the composition used was different from that of example 1 above, namely the polymer OTR4131 was in saline solution at a concentration of 10 µg/mL with 0.2% high-molecular-weight hyaluronic acid (HTL laboratories).

The patient was a 74-year-old man presenting with clinical signs of influenza, namely a high fever, approximately 39 degrees, difficulty and pain when breathing and fits of choking cough. As the patient was elderly, hospitalization was scheduled for 48 hours subject to the evolution of his clinical condition.

The patient was treated with a biocompatible polymer, namely the compound OTR4131, taking every two days 5 mL of a saline solution at 100 µg/mL of OTR4131 also comprising 0.2% hyaluronic acid.

In particular in this example the OTR4131 was used at 10 µ/mL in a 5 mg/mL solution of commercial HA (injectable grade).

The solution was administered orally by inhalation. The inhalation time was 10 minutes.

Surprisingly, 24 hours after the first dose of a composition comprising a biocompatible polymer according to the invention, namely OTR4131, the patient treated showed a significant reduction of his cough and also of his difficulty and pain when breathing.

A second dose of the aforementioned composition was given approximately 48 hours after the first dose. Confirming the observations made at 24 hours, surprisingly, 48 hours after the first dose, the treated patient showed a significant reduction of his cough and also of his difficulty and pain when breathing.

A clinical evaluation of the patient was carried out at 72 hours, the latter no longer showing signs of respiratory distress, an absence of pain and breathing difficulties, and an almost total disappearance of the cough.

All of the improvements, in particular at 24 hours after the first dose, were particularly linked to a treatment of lesions of the respiratory system caused by the virus, advantageously enabling an improvement and recovery of the functions of the respiratory system.

This example clearly demonstrates that examples of a composition according to the invention comprising a polymer of formula AaXxYy or AaXxYyZz and hyaluronic acid make it possible advantageously to treat and/or to improve treatment of lesions of the respiratory system caused by a microorganism, namely a virus, preferably lung lesions caused by a microorganism, in particular a virus. In particular, this example clearly demonstrates that examples of a composition according to the invention, particularly when administered by the pulmonary route, make it possible to treat lung lesions caused by viruses of the Orthomyxoviridae family, particularly the influenza virus.

This example also clearly demonstrates that examples of a composition according to the invention make it possible to treat lung lesions caused by a virus in a very short time. In particular, this example demonstrates surprisingly that the composition according to the invention comprising a polymer of formula AaXxYy or AaXxYyZz and hyaluronic acid makes it possible, for example in less than 72 hours to treat and/or significantly reduce the lesions of the respiratory system and/or of the lungs caused in particular by a virus and can advantageously make it possible to reduce the risk of associated morbidity.

This example also clearly demonstrates, through functional recovery, a recovery of lung function linked particularly to a restoration of the function of the alveolar-capillary barrier or air-blood barrier. In other words, an example of a composition according to the invention makes it possible both to treat lung lesions caused by a microorganism, in particular a virus, and also to restore lung function via restoration of the function of the alveolar-capillary barrier or air-blood barrier. Also, as demonstrated in this example, the present invention goes beyond the simple treatment of lung lesions and advantageously makes it possible, during a deficiency in the pulmonary function, for example respiratory distress, to synergistically restore the pulmonary function.

### Example 4: Use of a Biocompatible Polymer in the Treatment of a Patient One Month After Serious/Severe Infection with SARS-CoV-2

The patient was a 69-year-old radiologist infected by a patient with COVID-19 during a consultation for the analysis of images from a chest scan. The day of infection was therefore clearly identified as the day of the consultation. Four days later, the radiologist felt the first symptoms chills, temperature, joint pain, without loss of smell or taste. Ten days after infection and 6 days after the first symptoms, a PCR test for a diagnosis of SARS-CoV-2 infection was carried out, the result obtained was negative. A PCR test for a diagnosis of SARS-CoV-2 infection was performed 7 days after the first symptoms and this time showed a positive result with a value of 28 CT. A chest scan performed 7 days after the first symptoms revealed a “ground glass” lung surface around 20% associated with a lymph node nodule and vascular calcifications. Fifteen days after the first symptoms, the radiologist lost consciousness, his oxygen pressure fell to 96% and his fever remained constant at 38-39° C. with repeated losses of consciousness and systolic blood pressure falling between 9 and 9.4 mmHg. Blood tests, usual namely hematological, biochemical, hemostasis, were carried out 10 days, 13 days, 15 days, 17 days, 19 days, 23 days, 27 days, 32 days and 39 days post infection.

Only the tests showing differences with the normal according to the days are represented in table 2 below. The most visible parameters are C-reactive protein, ferritin and D-dimer

TABLE 2

Table of the radiologist’s blood and clinical parameter profile

| Days after infection | | 10 | 13 | 15 | 17 | 19 | 23 | 27 | 32 | 39 |
|---|---|---|---|---|---|---|---|---|---|---|
| Chest CT scan | | CT 1 | | | | | | | CT2 | |
| % lung lesions on the chest CT scan | | 20% | | | | | | | 50% | 10% |
| Compound: OTR4120 | | | | | | | | | Nebulization 5 minutes 3 mL at 10 µg/mL | |
| Biological parameters | Normal values | | | | | | | | | |
| LDH (lactate dehydrogenase) | 135-225 U/L | | 296 | | | | | | 296 | 242 |
| C-reactive protein (CRP) (mg/L) | <10 | 25.9 | 56.3 | 47.5 | 63.8 | 65.2 | 10 | | 3.3 | |
| Ferritin (µg/L) | 30-400 | 631.6 | | | | | | | | 366 |
| D-dimers (µg/l) | <500 | | <10 | 300 | | 400 | 597 | 597 | 359 | 358 |
| PCR COVID TEST | | Negative | CT 28 | | | Negative | | | | |
| Covid 19 IGM | | | | | | | | | | 7.1 |
| Covid 19 IGG | | | | | | | | | | 33.8 |

A chest scan was performed on the 32^{nd} day after infection before beginning treatment and showed a highly aggravated situation with approximately 50% lung damage. This state resulted particularly for the patient in deep fatigue and shortness of breath after walking for a few meters. The patient accustomed to walking an hour a day was unable to walk more than a few meters without shortness of breath and was barely leaving his home.

On the 32^{nd} day after infection, a treatment using an example of a composition according to the invention was initiated. The treatment consisted of nebulization using an ultrasonic or air jet nebulizer (common type found in pharmacies such as Omron, Newgen Medicale or equivalent whose prices vary between 40 and 200 euro). The composition used comprised OTR4120 at 100 µg/mL diluted in physiological saline solution (dilution 10 times in water) and poured into the diffusion chamber of the apparatus set to a flow rate of 0.5 mL/min. The patient received a total of 11 treatments/administrations/doses of 5 minutes, one morning and one evening per day.

From the first treatment/administration/dose given, the radiologist felt some tingling and a slight improvement in his breathing, which was particularly noticeable during the night. When he woke up he felt less tired than the previous days. The second dose/administration by nebulization confirmed this improvement in breathing and the next day (i.e. after 3 doses/administrations) the improvement was very clear both in terms of breathing and in terms of performance and/or the ability to move and tiredness. On the third day, the improvement was confirmed and the radiologist no longer felt shortness of breath or fatigue and resumed his walks. After the morning of the 5th day, i.e. after taking 11 doses/administrations, the radiologist stopped the nebulizations, he fully recovered his walking performance and no longer felt abnormal fatigue. No clinical signs related to SARS-CoV-2 were observable.

A blood test 8 days after beginning the treatment by nebulization showed normalization of concentrations/values.

A chest X-ray performed 17 days after the start of treatment revealed an 80% improvement in the lesion surface area of the lung, only 10% of the lesion surface was visible from a chest scan but was considered non-troublesome. Thus, as demonstrated, surprisingly a functional recovery of the respiratory function was obtained in a few days unlike untreated patients who need months in order to recover only part of their previous respiratory capacities from before the disease, or only part of their capacity causing lifelong sequelae.

This example therefore clearly demonstrates that an example of a composition according to the invention makes it possible to treat lung lesions caused by a virus, particularly a coronavirus, in particular SARS-CoV-2, in a very short time which can make it possible advantageously to reduce the risk of morbidity linked to lesions of the respiratory system and/or lungs caused in particular by viruses of the Coronaviridae family.

This example also clearly demonstrates, particularly in addition to a recovery of pulmonary function, an improvement in the general condition of the patient treated.

### Example 5: Use of a Biocompatible Polymer in a Prophylactic Treatment of SARS-CoV-2

In this example, an example of a composition according to the invention is used in a COVID-19 prevention application. Here two doctors assigned to Covid emergencies in two major Mexican hospitals used, under cover of confidentiality (to be verified/confirmed), OTR4120 in a 100 µg/mL solution marketed for the treatment of chronic wounds under the CACIPLIQ brand (registered trademark). The 5 mL solution was poured into a nose bulb making it possible to give a nasal spray or spray of approximately 100 µL each time the bulb is pressed. These doctors took a single spray per nostril per day for two months. While all the medical staff in each of these hospitals (i.e. around a hundred people per hospital) were infected with COVID-19 and actually fell ill (sometimes with severe forms and a few deaths), these two doctors were the only medical staff in each hospital not to have been affected by COVID-19.

This example therefore clearly demonstrates that an example of a composition according to the invention also has a prophylactic effect against COVID-19.

1. A pharmaceutical composition for use in the prophylaxis and/or treatment of lesions of the respiratory system caused by a microorganism, preferably lung lesions caused by a microorganism, said composition comprising
a biocompatible polymer of the following general formula (I) 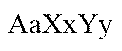
wherein:
A is a monomer,
X is an R₁COOR₂ or -R₉(C=O)R₁₀ group,
Y is an O or N-sulfonate group and has one of the following formulas -R₃OSO₃R₄, -R₅NSO₃R₆, R₇SO₃R₈ wherein:
R₁, R₃, Rs and R₉ are independently an aliphatic hydrocarbon chain, optionally branched and/or unsaturated and optionally containing one or more aromatic rings with the exception of benzylamine and benzylamine sulfonate, R₂, R₄, R₆ and R₈ are independently a hydrogen atom or an M⁺ cation,
R₇ and R₁₀ are independently a bond, an aliphatic hydrocarbon chain, optionally branched and/or unsaturated,
a is the number of monomers,
x is the rate of substitution of the A monomers by X groups,
y is the rate of substitution of the A monomers by Y groups.

2. A pharmaceutical composition for use in the treatment of deficiencies of respiratory functions due to lesions of the respiratory system caused by a microorganism, preferably lung lesions caused by a microorganism, said composition comprising
a biocompatible polymer of the following general formula (I) 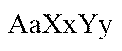
wherein:
A is a monomer,
X is an R₁COOR₂ or -R₉(C=O)R₁₀ group,
Y is an O or N-sulfonate group and has one of the following formulas -R₃OSO₃R₄, -R₅NSO₃R₆, R₇SO₃R₈ wherein:
R₁, R₃, R₅ and R₉ are independently an aliphatic hydrocarbon chain, optionally branched and/or unsaturated and optionally containing one or more aromatic rings with the exception of benzylamine and benzylamine sulfonate, R₂, R₄, R₆ and R₈ are independently a hydrogen atom or an M⁺ cation,
R₇ and R₁₀ are independently a bond, an aliphatic hydrocarbon chain, optionally branched and/or unsaturated,
a is the number of monomers,
x is the rate of substitution of the A monomers by X groups,
y is the rate of substitution of the A monomers by Y groups.

3. The composition to be used according to claim 1, wherein said composition additionally comprises hyaluronic acid.

4. The composition to be used according to claim 1 wherein the identical or different A monomers are selected from sugars, esters, alcohols, amino acids, nucleotides, nucleic acids, proteins or derivatives thereof.

5. The composition to be used according to claim 1 wherein the identical or different A monomers are selected from sugars or derivatives thereof.

6. The composition to be used according to claim 1 wherein the number of monomers “a” is such that the weight of said polymers of formula (I) is greater than or equal to 2000 Daltons.

7. The composition to be used according to claim 1 wherein the rate of substitution “x” is between 10 and 150%.

8. The composition to be used according to claim 1 wherein the rate of substitution “y” is between 10 and 170%.

9. The composition to be used according to claim 1 wherein said biocompatible polymer further comprises functional chemical groups Z, different from X and Y, capable of conferring to said polymer additional biological or physicochemical properties.

10. The composition to be used according to claim 9, wherein the rate of substitution “z” of all the A monomers by Z groups is between 1 and 50%.

11. The composition to be used according to claim 9, wherein the Z group is a substance capable of conferring to said polymers an improved solubility or lipophilicity.

12. The composition to be used according to claim 11, wherein the Z groups are identical or different and are selected from the group consisting of amino acids, fatty acids, fatty alcohols, ceramides, or derivatives thereof, or nucleotide addressing sequences.

13. The composition to be used according to claim 1 wherein the microorganism is selected from the group comprising viruses, bacteria and parasites.

14. The composition to be used according to claim 1 wherein the microorganism is a virus selected from the group comprising coronaviruses, rhinoviruses, influenza viruses.

15. The composition to be used according to claim 3 wherein the hyaluronic acid concentration is between 1 and 10 mg/mL.

16. The composition to be used according to claim 1 wherein the polymer concentration is between 0.1 and 100 µg/mL.
